(19) United States
(12) Patent Application Publication
(10) Pub. No.: US 2024/0148313 A1
(43) Pub. Date: May 9, 2024

(54) SYSTEM AND METHOD FOR ASSESSING CAPACITY FOR CONSCIOUSNESS

(71) Applicant: THE ROYAL INSTITUTION FOR THE ADVANCEMENT OF LEARNING/MCGILL UNIVERSITY, Montréal (CA)

(72) Inventors: Stefanie BLAIN-MORAES, Montreal West (CA); Catherine DUCLOS, Montréal (CA)

(21) Appl. No.: 17/981,195

(22) Filed: Nov. 4, 2022

(51) Int. Cl. A61B 5/377; A61B 5/372; G16H 50/30

(52) U.S. Cl. CPC A61B 5/377; A61B 5/372; G16H 50/30

(57) ABSTRACT

There is provided a system and a method for determining a capacity for consciousness of a person. Neuroimaging data acquired by a neuroimaging system before, during, and following an exposure of the person to a neurophysiological perturbation agent having anesthetic properties is obtained. From the neuroimaging data, a degree of reconfiguration exhibited by a functional brain network of the person as a result of the exposure to the neurophysiological perturbation agent is determined. A measure of the capacity for consciousness of the person is computed based on the degree of reconfiguration exhibited by the functional brain network.

### FIELD

The improvements generally relate to the field of predicting patient outcome, and more specifically to assessing a patient's capacity for consciousness.

### BACKGROUND

Detecting covert consciousness and predicting recovery in unresponsive, brain-injured individuals remains an important shortcoming of clinical practice, with crucial implications for clinical management and decision-making. Most current techniques for the diagnosis of conscious awareness and the prognosis of consciousness recovery are limited by their reliance on a patient's ability and willingness to respond to commands/stimuli or the requirement of expensive medical equipment that is difficult to access at bedside. In particular, the majority of current techniques rely on specialized technologies, such as magnetic resonance imaging (MRI) and positron-emission tomography (PET), which have contraindications that exclude many patients suffering from a disorder of consciousness (DOC), and are challenging to integrate into everyday clinical environments, preventing their widespread adoption for the assessment of DOC patients.

Therefore, there remains a need for improvement.

### SUMMARY

In accordance with one aspect, there is provided a method for determining a capacity for consciousness of a person. The method comprises obtaining neuroimaging data acquired by a neuroimaging system before, during, and following an exposure of the person to a neurophysiological perturbation agent having anesthetic properties, determining, from the neuroimaging data, a degree of reconfiguration exhibited by a functional brain network of the person as a result of the exposure to the neurophysiological perturbation agent, and computing a measure of the capacity for consciousness of the person based on the degree of reconfiguration exhibited by the functional brain network.

In accordance with another aspect, there is provided a system for determining a capacity for consciousness of a person. The system comprises a processing unit and a non-transitory computer-readable memory communicatively coupled to the processing unit and comprising computer-readable program instructions executable by the processing unit for obtaining neuroimaging data acquired by a neuroimaging system before, during, and following an exposure of the person to a neurophysiological perturbation agent having anesthetic properties, determining, from the neuroimaging data, a degree of reconfiguration exhibited by a functional brain network of the person as a result of the exposure to the neurophysiological perturbation agent, and computing a measure of the capacity for consciousness of the person based on the degree of reconfiguration exhibited by the functional brain network.

In accordance with another aspect, there is provided a non-transitory computer readable medium having stored thereon program code executable by at least one processor for obtaining neuroimaging data acquired by a neuroimaging system before, during, and following an exposure of a person to a neurophysiological perturbation agent having anesthetic properties, determining, from the neuroimaging data, a degree of reconfiguration exhibited by a functional brain network of the person as a result of the exposure to the neurophysiological perturbation agent, and computing a measure of the capacity for consciousness of the person based on the degree of reconfiguration exhibited by the functional brain network of the person.

Many further features and combinations thereof concerning embodiments described herein will appear to those skilled in the art following a reading of the instant disclosure.

In the figures,

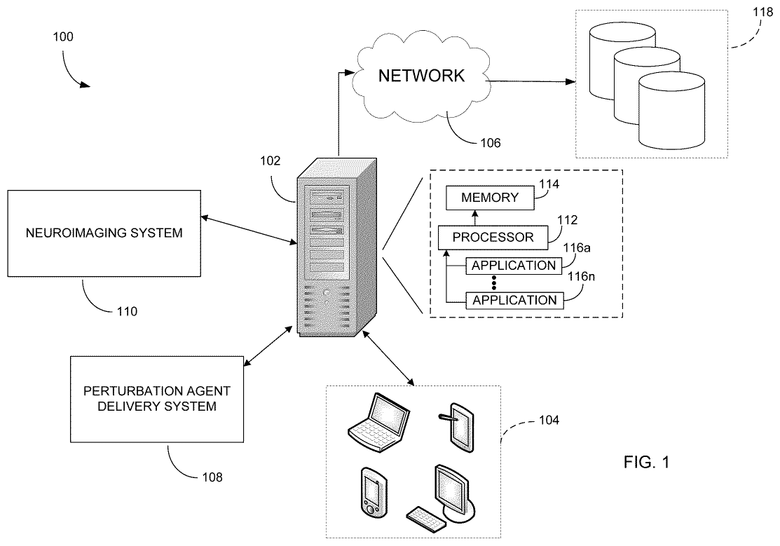

FIG. 1 is a schematic diagram of a system for determining a person's capacity for consciousness, in accordance with an illustrative embodiment;

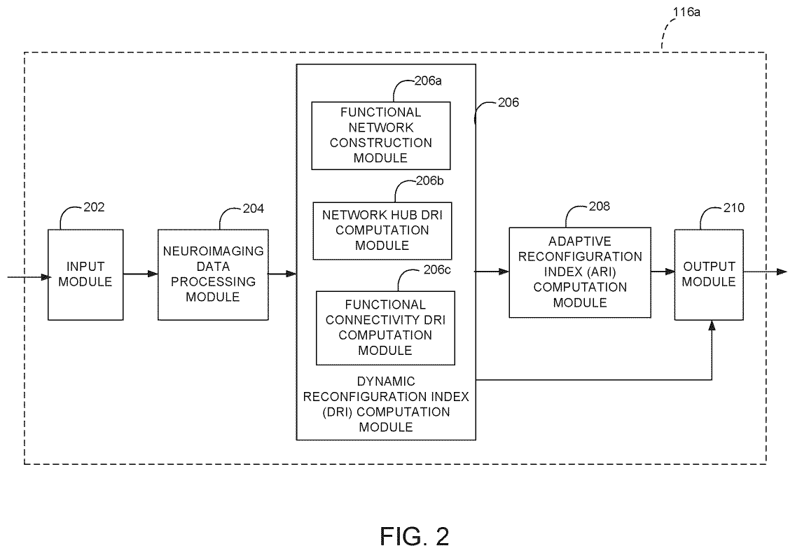

FIG. 2 is a schematic diagram of an application running on the processor of FIG. 1, in accordance with an illustrative embodiment;

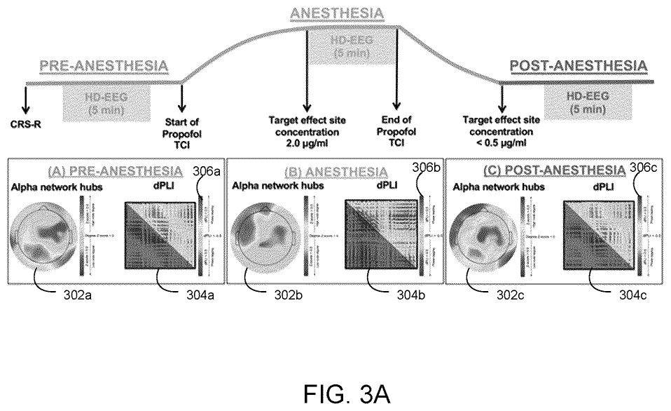

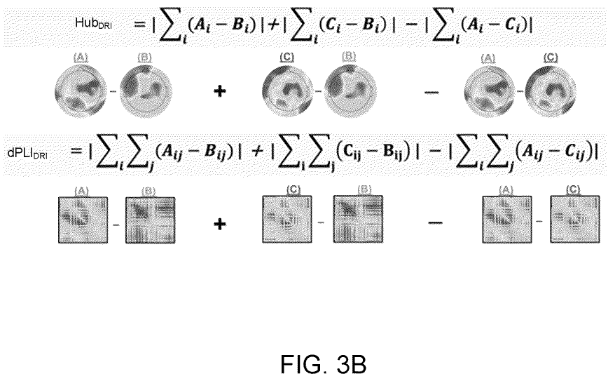

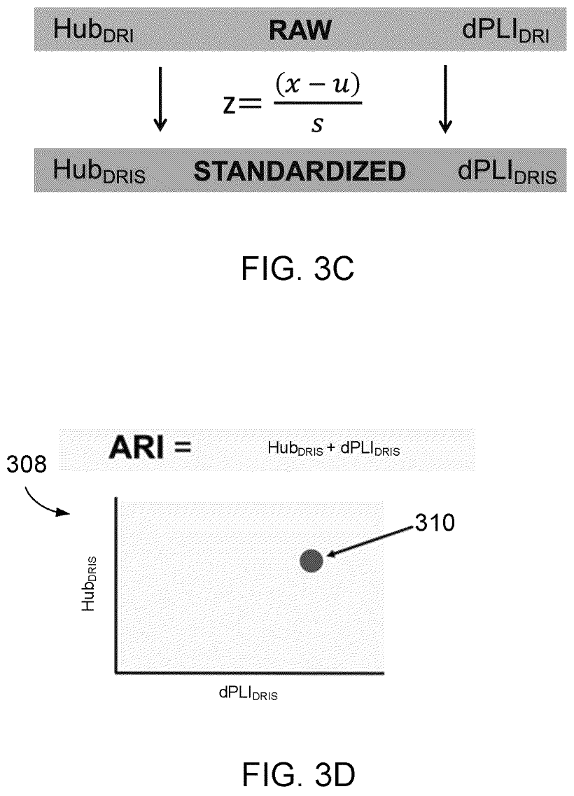

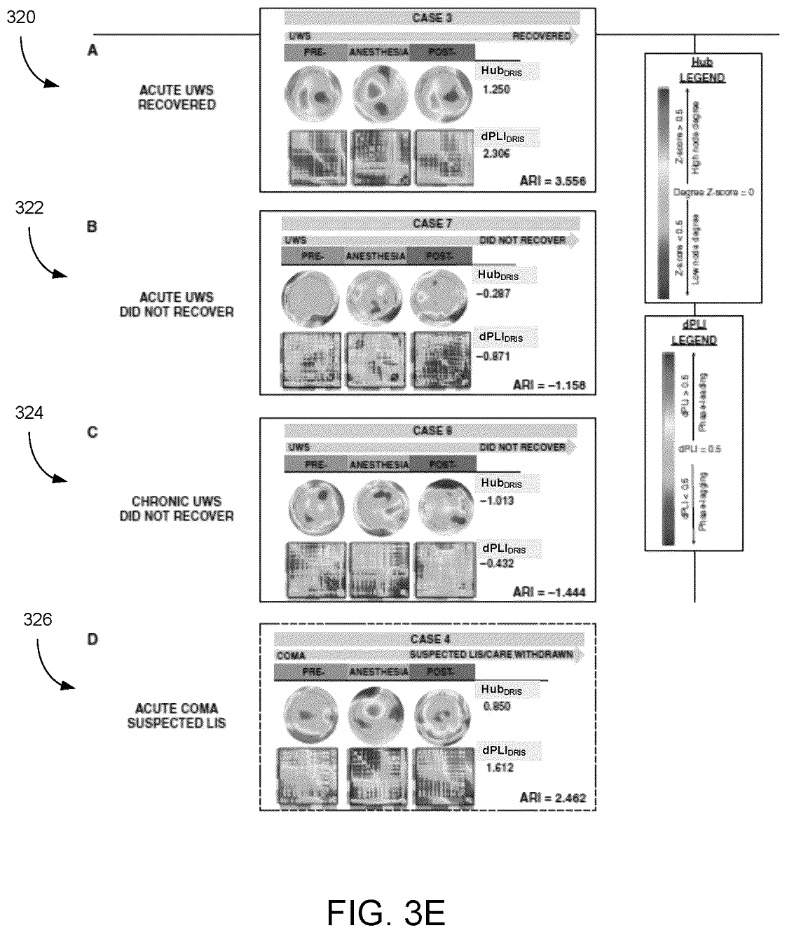

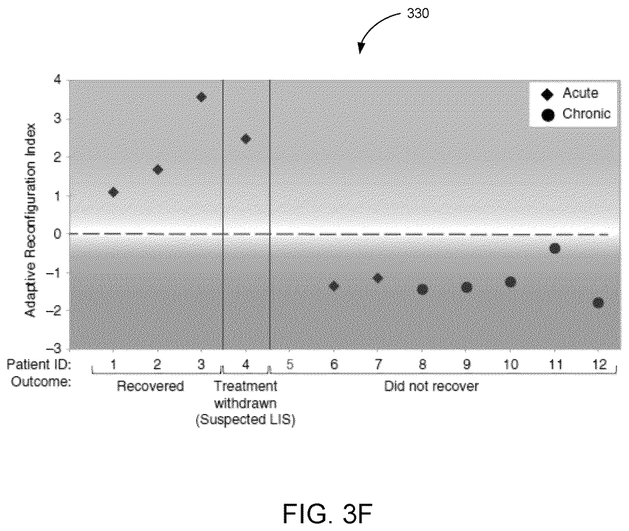

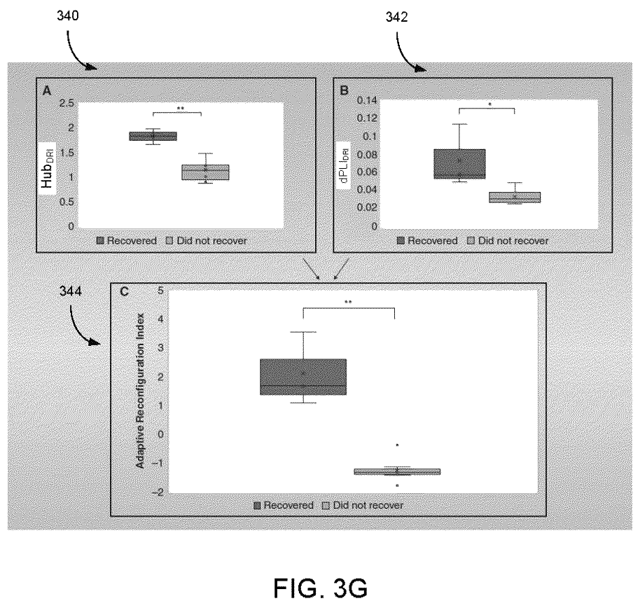

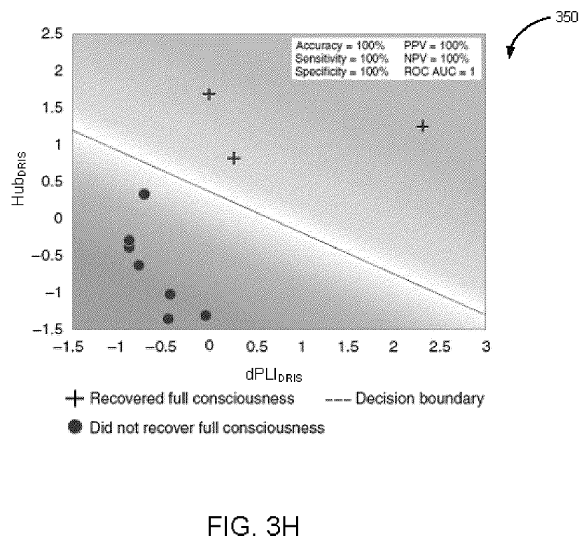

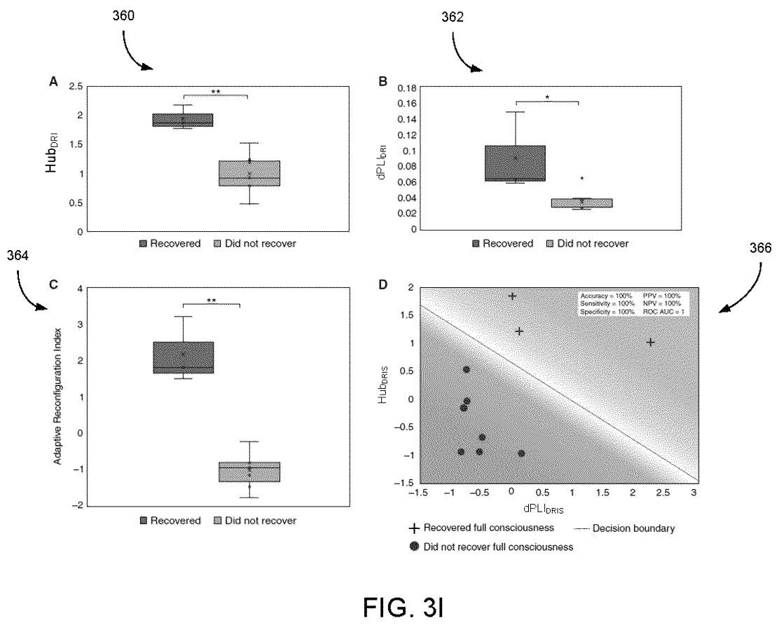

FIG. 3A is a schematic diagram illustrating an anesthetia protocol administration process and a neuroimaging recording process implemented by the system of FIG. 1, in accordance with an illustrative embodiment;

FIG. 3B is a schematic diagram illustrating a neuroimaging feature extraction process implemented by the system of FIG. 1, in accordance with an illustrative embodiment;

FIG. 3C is a schematic diagram illustrating a data standardization process implemented by the system of FIG. 1, in accordance with an illustrative embodiment;

FIG. 3D is a schematic diagram illustrating computation of an Adaptive Reconfiguration Index (ARI) implemented by the system of FIG. 1, in accordance with an illustrative embodiment;

FIGS. 3E to 3I illustrate ARI computation results, in accordance with an illustrative embodiment;

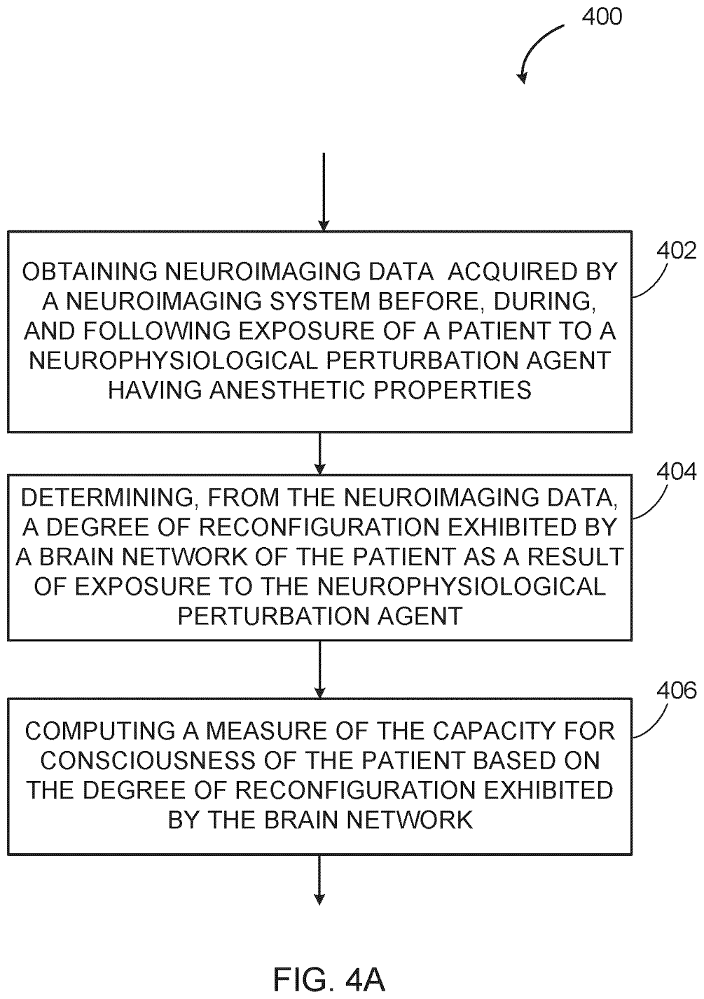

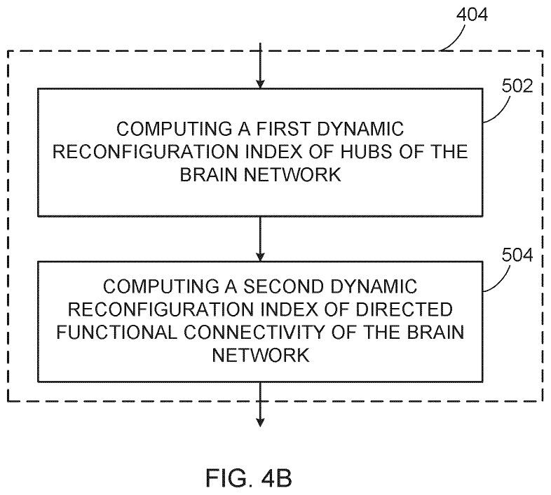

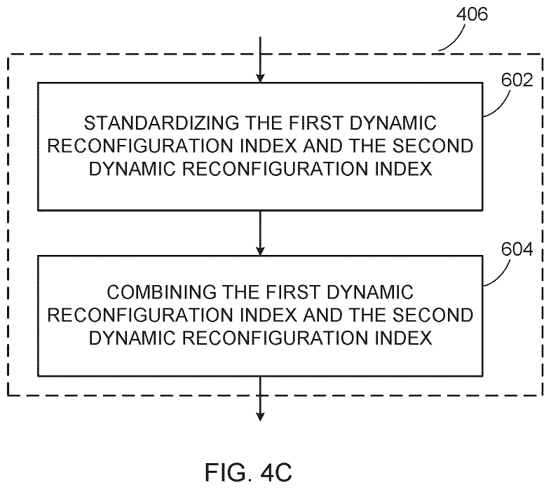

FIG. 4A is a schematic diagram of a method for determining a person's capacity for consciousness, in accordance with an illustrative embodiment;

FIG. 4B is a schematic diagram of the step of FIG. 4A of determining a degree of reconfiguration exhibited by a functional brain network, in accordance with an illustrative embodiment; and

FIG. 4C is a schematic diagram of the step of FIG. 4A of computing a measure of the capacity for consciousness of a person, in accordance with an illustrative embodiment.

It will be noted that throughout the appended drawings, like features are identified by like reference numerals.

### DETAILED DESCRIPTION

After brain injury, many patients are left in an unresponsive state, making it difficult to predict their eventual recovery. This has significant implications for clinical decision making, information provided to families, and optimal use of healthcare resources. Proposed herein are systems and methods that use a translation index (referred to herein as an adaptive reconfiguration index or ARI), in the assessment and prognostication of conscious awareness in the absence of behavioral responsiveness.

As will be described further below, the ARI is an empirical prognostic measure of brain adaptability to predict recovery of consciousness in unresponsive, brain-injured individuals. A neuroimaging modality, such as electroencephalography (EEG), is used to measure the response of a person's (e.g., a patient's) brain before, during, and after a neurophysiological perturbation. This is achieved by using scalp electrodes to measure the electrical activity of cortical neurons in the patient's functional brain network. In one embodiment, it is proposed herein to use the intravenous general anesthetic Propofol as the neurophysiological perturbation agent. The advantages of using Propofol include its ubiquity across acute care settings, its familiarity for critical care healthcare specialists, and its relative low cost make. As a result, in one embodiment, the use of Propofol to determine the ARI makes the systems and methods described herein translational and facilitates their integration into the treatment of disorder of consciousness (DOC) patients. It should however be understood that the ARI may be determined by measuring the brain's response to neurophysiological perturbations other than Propofol. Any perturbation agent having anesthetic properties suitable to make a human lose unconsciousness may be used. For example, anesthetic agents or compounds including, but not limited to, Sevoflurane, Isoflurane, Dexmedetomidine, and Ketamine, may apply.

In one embodiment and as will be discussed further below, the ARI is calculated by (i) perturbing the brain with a targeted dose of Propofol anesthesia and (ii) contrasting functional brain network properties before, during and after exposure to anesthesia. Unlike existing prognostic measures that rely on global or event-related brain signals, the systems and methods described herein focus on the brain signals that are attenuated by the effects of the general anesthesia, which putatively include those associated with conscious awareness. Indeed, the healthy brain undergoes an organized functional reconfiguration as it loses consciousness in response to exposure to the anesthetic perturbation agent (e.g., Propofol). The proposed systems and methods described herein are based on the hypothesis that unresponsive, brain-injured patients who undergo these network reconfigurations in response to the anesthetic perturbation agent, indicating the loss of some residual consciousness, currently possess consciousness, despite being unresponsive, and/or have the capacity to recover.

In one embodiment, to determine the ARI, the patient's functional brain network properties being contrasted are network hubs (and particularly anteriorization of alpha network hubs) and directed phase-based functional connectivity (and particularly neutralization of feedback-dominant connectivity). As known to those skilled in the art, network hubs are densely connected nodes within a functional brain network. In particular, network hubs are nodes that occupy a central position in the overall organization of the functional brain network. They are parts of the brain that make connections with other parts of the brain. In healthy conscious individuals, alpha network hubs are located in the posterior regions of the brain. As also known to those skilled in the art, functional connectivity represents the connectivity between brain regions that share functional properties. Resting-state functional connectivity of the functional brain network reflects the inherent and spontaneous neural activity of the brain activity pattern. Directed phase-based functional connectivity is specifically affected by Propofol anesthesia. This reconfiguration of the brain's functional network, which is induced by Propofol anesthesia (or any other suitable perturbation agent), is then translated into the ARI.

A high ARI is indicative of favorable prognosis (i.e. recovery of full consciousness), such that the ARI is expected to be higher in DOC patients with the capacity for consciousness, compared to DOC patient without capacity for consciousness. This is due to the fact that a brain that has the capacity for consciousness will undergo network alterations in response to anesthesia and will revert to its pre-anesthesia state when anesthesia is stopped. Thus, patients with the capacity to recover full consciousness will exhibit greater brain reconfiguration (i.e. network hub reconfiguration and directed functional connectivity reconfiguration) in response to the perturbation agent (i.e. anesthesia) than patients without the capacity to recover. The ARI is therefore low when there is little change in network configuration upon exposure to the perturbation agent, and when functional brain networks do not return to their baseline configuration after exposure to the perturbation agent. This is the case, for example, with chronic DOC patients. In other words, it is the inability for the patient's functional brain network patterns to reconfigure upon exposure to the perturbation agent that reflects the patient's capacity for recovery.

Referring now to FIG. 1, a system 100 for assessing (or determining) a person's (e.g., a patient's) capacity for consciousness will now be described, in accordance with one embodiment. The illustrated system 100 comprises one or more server(s) 102 adapted to communicate with a plurality of devices 104 as well as over a network 106, such as the Internet, a cellular network, Wi-Fi, or others known to those skilled in the art. The devices 104 allow users, such as physicians and other healthcare professionals, to gain access to the system 100. The devices 104 may comprise any suitable device (whether mobile or not) and may be configured to access the network 106 via the server(s) 102, for example to access data stored in one or more databases 118. Examples of the devices 104 include, but are not limited to, laptop computers, desktop personal computers, handled personal computers, tablet computers, and smartphones. The devices 104 may run a browsing program, such as Microsoft's Internet Explorer™, Safari™, a Wireless Application Protocol (WAP) enabled browser in the case of a smart phone, or a native mobile application. Each device 104 may also include an input/output interface (not shown) that enables the device to interconnect with one or more input devices (not shown), such as a keyboard, a mouse, a touchscreen, a camera, a microphone, and the like, or with one or more output devices (not shown), such as a display screen, a speaker, and the like.

The system 100 further comprises a perturbation agent delivery system 108, which is configured to administer to a patient a perturbation agent having anesthetic properties. In one embodiment and as described herein above, the perturbation agent delivery system 108 is configured to administer a stable and targeted concentration of Propofol. The administration information (e.g., dose, rate, and the like) associated with the perturbation agent is determined based on the patient's unique profile (e.g., gender, age, weight, height, and the like), such that the anesthetia protocol may vary from one patient to the next. In some embodiments, the server 102 may be configured to output one or more control signal(s) to the perturbation agent delivery system 108 to cause administration of the perturbation agent to the patient in accordance with the administration information.

The system 100 also comprises a neuroimaging system 110. In one embodiment, the neuroimaging system 110 is an EEG recording system, which is used to detect a patient's EEG signatures (i.e. monitor the patient's physiological response) as a result of the patient's exposure to the perturbation agent (e.g., Propofol anesthesia). Such an EEG recording system illustratively comprises an EEG electrode array comprising frontal electrodes, central electrodes, parietal electrodes, occipital electrodes, and temporal electrodes positioned on the patient's scalp surface for acquiring raw EEG signals. The EEG electrode array may comprise any suitable number of electrode channels. For example, a 64-channel electrode EEG system or a 128-channel electrode EEG system may be used. In one embodiment, electrode impedances may be kept below 50 kΩ. In one embodiment, the EEG recording system uses high-density EEG (hd-EEG) technology to acquire resting-state EEG signals (i.e. EEG signals acquired during task-free spontaneous brain activity). The EEG signals are acquired over a pre-determined timeframe (e.g., five (5) minutes) at baseline (i.e. pre-anesthesia), during exposure to Propofol anesthesia at a stable concentration, and after recovery from anesthesia (i.e. post-anesthesia). The EEG recording system then sends the acquired EEG signals to the server 102 for subsequent analysis and use in determining the ARI.

While the neuroimaging system 110 is described herein as an EEG recording system for illustrative purposes, it should be understood that any other neuroimaging system from which a functional brain network can be constructed may apply. For example, the neuroimaging system 110 may use other neuroimaging modalities including, but not limited to, functional magnetic resonance imaging (fMRI) and functional near infrared spectroscopy (fNIRS). Data (referred to herein as “neuroimaging data”) acquired by the neuroimaging system 110 may then be sent to the server 102 for use in determining the ARI.

The server 102 may comprise a series of servers corresponding to a web server, an application server, and a database server. These servers are all represented by server 102 in FIG. 1. The server 102 may comprise, amongst other things, a processor 112 coupled to a memory 114 and having a plurality of applications 116a, . . . , 116n running thereon. The processor 112 may access the memory 114 to retrieve data. The processor 112 may be any device that can perform operations on data. Examples are a central processing unit (CPU), a microprocessor, and a front-end processor. The applications 116a, . . . , 116n are coupled to the processor 112 and configured to perform various tasks as explained below in more detail. It should be understood that while the applications 116a, . . . , 116n presented herein are illustrated and described as separate entities, they may be combined or separated in a variety of ways. It should be understood that an operating system (not shown) may be used as an intermediary between the processor 112 and the applications 116a, . . . , 116n.

The memory 114 accessible by the processor 112 may receive and store data. The memory 114 may be a main memory, such as a high speed Random Access Memory (RAM), or an auxiliary storage unit, such as a hard disk or flash memory. The memory 114 may be any other type of memory, such as a Read-Only Memory (ROM), Erasable Programmable Read-Only Memory (EPROM), or optical storage media. Also, although the system 100 is described herein as comprising the processor 112 having the applications 116a, . . . , 116n running thereon, it should be understood that cloud computing may also be used. As such, the memory 114 may comprise cloud storage.

The one or more databases 118 may be integrated directly into the memory 114 or may be provided separately therefrom and remotely from the server 102 (as illustrated). In the case of a remote access to the databases 118, access may occur via any type of network 106, as indicated above. The databases 118 described herein may be provided as collections of data or information organized for rapid search and retrieval by a computer. The databases may be structured to facilitate storage, retrieval, modification, and deletion of data in conjunction with various data-processing operations. The databases 118 may consist of a file or sets of files that can be broken down into records, each of which consists of one or more fields. Database information may be retrieved through queries using keywords and sorting commands, in order to rapidly search, rearrange, group, and select the field. The databases may be any organization of data on a data storage medium, such as one or more servers. As discussed above, the system 100 may use cloud computing and it should therefore be understood that the databases 118 may comprise cloud storage.

In one embodiment, the databases 118 are secure web servers and Hypertext Transport Protocol Secure (HTTPS) capable of supporting Transport Layer Security (TLS), which is a protocol used for access to the data. Communications to and from the secure web servers may be secured using Secure Sockets Layer (SSL). Identity verification of a user may be performed using usernames and passwords for all users. Various levels of access authorizations may be provided to multiple levels of users.

Alternatively, any known communication protocols that enable devices within a computer network to exchange information may be used. Examples of protocols are as follows: IP (Internet Protocol), UDP (User Datagram Protocol), TCP (Transmission Control Protocol), DHCP (Dynamic Host Configuration Protocol), HTTP (Hypertext Transfer Protocol), FTP (File Transfer Protocol), Telnet (Telnet Remote Protocol), SSH (Secure Shell Remote Protocol).

Referring now to FIG. 2 in addition to FIG. 1, an exemplary embodiment of an application 116a running on the processor 112 will now be described. The application 116a illustratively comprises an input module 202 and a neuroimaging data processing module 204. The application 116a also comprises a dynamic reconfiguration index (DRI) computation module 206 comprising a functional network construction module 206a, a network hub DRI computation module 206b, and a functional connectivity DRI computation module 206c. The application 116a further comprises an adaptive reconfiguration index (ARI) computation module 208 and an output module 210.

The input module 202 is configured to receive neuroimaging data from the neuroimaging system 110. As described herein above, in one embodiment, the neuroimaging data comprises EEG signals collected from the patient's scalp using an electrode array. The neuroimaging data is then sent from the input module 202 to the neuroimaging data processing module 204, which is configured to process the neuroimaging data for subsequent analysis. In one embodiment, processing of the neuroimaging data comprises bandpass filtering the collected EEG signals and discarding non-scalp channels. In one embodiment, the EEG signals are bandpass filtered within a frequency range from 0.1 Hz to 50 Hz. Preprocessing of the EEG signals may also comprise removing noisy epochs and channels, as well as muscle and non-physiological artifacts from the EEG signals. The neuroimaging data processing module 204 is further configured to re-reference the processed neuroimaging data to an average reference. Segments of neuroimaging data of a given duration (e.g., five-minute segments) are then extracted during three analysis epochs: pre-anesthesia, anesthesia, and post-anesthesia. The extracted neuroimaging data segments are then sent to the DRI computation module 206 for use in computing the ARI.

In particular, the DRI computation module 206 is configured to compute, using the network hub DRI computation module 206b, the dynamic reconfiguration index of network hubs (Hub_{DRI}) before, during, and after anesthetic exposure, and to compute, using the functional connectivity DRI computation module 206c, the dynamic reconfiguration of directed functional connectivity (dPLI_{DRI}) by contrasting connectivity matrices before, during, and after anesthetic exposure. For this purpose, the DRI computation module 206 first uses the functional network construction module 206a to construct functional networks. This may be achieved using a weighted phase lag index (wPLI) of all pairwise combinations of electrode channels. In one embodiment, the wPLI is calculated across 10-second windows and averaged within each analysis epoch in the alpha (8-14 Hz) frequency band to generate representative connectivity matrices for pre-anesthesia, anesthesia, and post-anesthesia periods.

The wPLI between two electrode channels is computed as:

$${wPLI}_{ij} = \frac{\vert E \{ \tilde{𝓈} ( {C}_{ij} ) \} \vert}{E \{ \vert \tilde{𝓈} ( {C}_{ij} ) \vert \}} = \frac{\vert E \{ \vert \tilde{𝓈} ( {C}_{ij} ) \vert sgn ( \tilde{𝓈} ( {C}_{ij} ) ) \} \vert}{E \{ \vert \tilde{𝓈} ( {C}_{ij} ) \vert \}} \qquad ( 1 )$$

ℑ(Cᵢⱼ) where is the imaginary part of the cross-spectrum Cᵢⱼ between i and j, and sgn is the signum function.

When one signal leads the other, the wPLI is close to 1, with a value of 1 indicating perfect phase locking between signals. When there is no phase relationship between the signals, the wPLI is equal to 0. In one embodiment, the effects of spurious phase relationships may be controlled through a surrogate analysis, where signal i remains fixed while the phase time-series of signal j is scrambled, abolishing the phase relationship between the signals while maintaining their other properties. The wPLI may then be compared against a distribution of the means of 20 surrogate analyses. wPLI values may then be retained if they are significantly different than the surrogate distribution (p<0.05 level).

The functional network construction module 206a then constructs a binary adjacency matrix Aij using a custom threshold for each patient. If the wPLIij value of nodes i and j is above the custom threshold of all wPLI values, Aij=1; otherwise, Aij=0. In one embodiment, the custom threshold may be determined for each patient by identifying the lowest threshold enabling a minimally-spanning graph during the pre-anesthesia recording. The threshold established for the pre-anesthesia network may also be used for constructing the functional networks in the anesthesia and post-anesthesia phases. The degree of (i.e. the number of network connections, or edges, attached to) each node in each functional network is then calculated to assess the location of high-degree network hubs across each analysis epoch. The functional network construction module 206a may then generate a map of the topographic distribution of hubs based on the degree z-score of each node within a given network relative to all the other nodes in the network. The topographic map may be sent from the DRI computation module 206 to the output module 210 for rendering on an output device (e.g., display on a screen) associated with the devices 104.

FIG. 3A is a schematic diagram illustrating an anesthetia protocol administration process and a neuroimaging (e.g., EEG) recording process implemented by the system 100 of FIG. 1, in accordance with one embodiment. FIG. 3A shows topographic maps 302a, 302b, 302c generated for pre-anesthesia, anesthesia, and post-anesthesia periods, respectively. In one embodiment, each topographic map 302a, 302b, 302c representing the z-score of the normalized node degree of each electrode.

The functional network construction module 206a further calculates the directed phase lag index (dPLI) across predetermined timeframes (e.g., 10-second windows) and averages the dPLI within each analysis epoch in the alpha frequency band to generate representative directed functional connectivity matrices for pre-anesthesia, anesthesia, and post-anesthesia periods. The functional connectivity matrices may be sent from the DRI computation module 206 to the output module 210 for rendering on the devices 104. FIG. 3A illustrates functional connectivity matrices 304a, 304b, 304c generated for pre-anesthesia, anesthesia, and post-anesthesia periods, respectively. In one embodiment, each functional connectivity matrix 304a, 304b, 304c depicts a single brain hemisphere for the patient for visualization purposes. In cases of focal lesions, the depicted brain hemisphere is the hemisphere with the least severe neuronal damage. In cases of diffused brain injury, the depicted brain hemisphere is the hemisphere with the healthiest reconfiguration pattern. It should however be understood that, in other embodiments, both brain hemispheres may be depicted and considered. In one embodiment, electrodes are ordered per region and represented by a colorbar 306a, 306b, 306c bordering each matrix, with a color being assigned to each one of the frontal electrodes, central electrodes, parietal electrodes, occipital electrodes, and temporal electrodes. In one embodiment, each mapped functional connectivity matrix 304a, 304b, 304c represents the strength of lead-lag relationships for each electrode pair, with one color depicting phase-leading, and another color depicting phase-lagging.

A Hilbert transform may be used to yield the instantaneous phase time series for each channel, and the phase difference Δϕij between all pairs of signals i and j may be calculated. Directed functional connectivity is calculated with the dPLI defined as:

$${dPLI}_{ij} = \frac{1}{N} {\sum }_{t = 1}^{N} H ( {Δϕ}_{ij} ) \qquad ( 2 )$$

where N is the length of the analysis segment and t is a given time point. H is the Heaviside step function, such that when i leads j, the dPLI is between 0.5 and 1; when j leads i, the dPLI is between 0 and 0.5; and, when there is no phase relationship between the signals, the dPLI is equal to 0.5. The dPLI values may be compared against a distribution of the means of 20 surrogate analyses. As described above, each surrogate signal i remains fixed while the phase time-series of signal j is scrambled, abolishing the phase relationship between the signals while maintaining their other properties. dPLI values are then retained if they are significantly different than the surrogate distribution (p<0.05 level), and non-significant connections ae set to 0.5.

FIG. 3B is a schematic diagram illustrating a neuroimaging (e.g., EEG) feature extraction process implemented by the system 100 of FIG. 1, in accordance with one embodiment. As illustrated in FIG. 3B, the reconfiguration of network hubs across pre-anesthesia, anesthesia, and post-anesthesia phases is quantified using the network hub DRI 340 computation module 206b, which calculates a dynamic reconfiguration index (DRI) of network hubs (or Hub_{DRI}). For this purpose, differences in node degree across phases is calculated as follows:

Hub_{DRI}=Σᵢ(Aᵢ−Bᵢ)+Σᵢ(Cᵢ−Bᵢ)−Σᵢ(Aᵢ−Cᵢ) (3)

where A is a vector of network degree (i.e. a vector indicating the degree of each node in the functional brain network) pre-anesthesia, B is a vector of network degree during anesthesia, and C is a vector of network degree post-anesthesia, and where i is the index associated with (i.e. corresponding to) a given electrode. In networks with high reconfiguration in response to anesthesia, Σ(Ai−Bi)i and Σ(Ci−Bi)i are expected to be high, as they contrast anesthesia with a non-anesthetic phases, whereas Σ(Ai−Ci)i is expected to be low, as it contrasts pre- and post-anesthesia phases. High Hub_{DRI} is indicative of higher network topographic reconfiguration across the three phases, where low Hub_{DRI} reflects limited reconfiguration across phases.

As also illustrated in FIG. 3B, the reconfiguration of directed functional connectivity across pre-anesthesia, anesthesia, and post-anesthesia phases is quantified using the functional connectivity DRI computation module 206c, which calculates a DRI of dPLI (or dPLI_{DRI}). This is achieved by calculating differences in node degree across phases, as follows:

dPLI_{DRI}=ΣᵢΣⱼ(Aᵢⱼ−Bᵢⱼ)+ΣᵢΣⱼ(Cᵢⱼ−Bᵢⱼ)−ΣᵢΣⱼ(Aᵢⱼ−Cᵢⱼ) (4)
where A is the dPLI matrix pre-anesthesia, B is the dPLI matrix during anesthesia, and C is the dPLI matrix post-anesthesia, and i and j are indices associated with (i.e. representing) individual electrodes. In a network with high reconfiguration in response to anesthesia, ΣΣ(Aij−Bij)ji and ΣΣ(Cij−Bij))ji are expected to be high, and the difference between non-anesthesia phases, ΣΣ(Aij−Cij)ji is expected to be low. High dPLI_{DRI} is indicative of higher reconfiguration of directed connectivity across the three phases, where low dPLI_{DRI} reflects limited reconfiguration of directed functional connectivity across phases. Hub_{DRI} and dPLI_{DRI} are then standardized (at the network hub DRI computation module 206b and the functional connectivity DRI computation module 206c, respectively), becoming Hub_{DRIS} and dPLI_{DRIS}. As used herein, the term “standardization” refers to data processing technique implemented to bring (i.e. convert the structure of) disparate datasets into a common data format, thereby allowing the datasets to be compared with one another. Any suitable technique may be used. In one embodiment, the data standardization process is achieved using the Scikit-learn implementation, by, for each of Hub_{DRI} and dPLI_{DRI}, removing the mean and scaling to unit variance, as illustrated in FIG. 3C. This data standardization process ensures both variables have the same weight in the algorithm, despite varying scales in their unstandardized format. The standardized Hub_{DRI} (i.e. Hub_{DRIS}) and the standardized 375 dPLI_{DRI} (i.e. dPLI_{DRIS}) are then sent from the DRI computation module 206 to the ARI computation module 208. The ARI computation module 208 then computes the ARI as the combination of the standardized Hub_{DRI} (Hub_{DRIS}) and the standardized dPLI_{DRI} (dPLI_{DRIS}). In particular, the ARI is computed as a single-digit index which is the sum of Hub_{DRIS} and dPLI_{DRIS} and represents the amount of topographic reconfiguration exhibited by functional brain 380 networks when perturbed by anesthesia. In other words, the ARI reflects the adaptive reconfiguration of functional brain networks across pre-anesthesia, anesthesia, and post-anesthesia phases. The ARI computed by the ARI computation module 208 is then sent to the output module 210 for rendering on the devices 104.

Although reference is made herein to the ARI being computed (e.g., by the ARI 385 computation module 208) as the combination of the standardized Hub_{DRI} (Hub_{DRIS}) and the standardized dPLI_{DRI} (dPLI_{DRIS}), it should be understood that additional functional network measures including, but not limited to, power-law distribution and markers of criticality, may also apply.

Any suitable visual representation of the ARI and its two components (Hub_{DRIS} and dPLI_{DRIS}) may be used. In one embodiment, the ARI is rendered in a two-dimensional space as a plot of Hub_{DRIS} as a function of dPLI_{DRIS}. FIG. 3D is a schematic diagram illustrating computation of the ARI, as implemented by the system 100 of FIG. 1, in accordance with an illustrative embodiment. FIG. 3D illustrates a plot 308 of Hub_{DRIS} (y-axis) and dPLI_{DRIS} (x-axis) in the two-dimensional feature space for a given patient, yielding the ARI value 310 per patient.

In order to appraise the diagnostic and prognostic value of ARI among individuals with a DOC, the systems and methods proposed herein were tested in a sample of twelve (12) individuals with a disorder of consciousness (DOC), e.g. after a brain injury. Patients in a coma were in a deep state of unconsciousness, lacking both wakefulness and awareness, and had no responses to stimulation and pain. Patients with a disorder of consciousness had preserver ability to awaken but no confirmed signs of awareness. These patients had unresponsive wakefulness syndrome (also referred to as a vegetative state) or a minimally conscious state. With unresponsive wakefulness syndrome, eye opening is present but patients show no behavioral signs of being aware of themselves or their surroundings, lacking oriented or willful behaviors. Patients with unresponsive wakefulness syndrome are therefore considered to be unconscious. A minimally conscious state presents with eye opening and some reproducible but minimal oriented and/or willful behaviors (e.g., visual tracking and inconsistent command following).

As illustrated in FIG. 3A, participants were given Propofol in target-infusion mode (also referred to as target-controlled infusion or TCI) at predicted target effect-site concentration of 2.0 μg/mL using the Marsh pharmacokinetic model. Resting-state hd-EEG signals were acquired for five (5) minutes at baseline (pre-anesthesia), during exposure to Propofol anesthesia (anesthesia), and after recovery from anesthesia (post-anesthesia). EEG signals were collected from the scalp using a 128-channel or 64-channel electrode set. The patients' current level of consciousness was then assessed using the Coma Recovery Scale-Revised (CRS-R), immediately preceding the anesthetia protocol. After three (3) months, participants were deemed to have recovered full consciousness if they were able to consistently follow commands and/or respond verbally in an appropriate manner to conversation (i.e., if functional/accurate communication or functional object use was present, denoting emergence from DOC, as per CRS-R criteria). It should however be understood that the ARI may be used to determine a capacity of consciousness of a patient based on a level of responsiveness over any suitable time period other than three (3) months (e.g., years post-injury) and that any suitable classification of patients other than “recovered” or “non-recovered” may apply.

The association between ARI and current level of consciousness (i.e. whether some signs of consciousness are present or not) as well as recovery of full consciousness (i.e. whether responsiveness was recovered or not) was also investigated. One-tailed Mann-Whitney U-tests were conducted to determine if the ARI and its components (Hub_{DRI} and dPLI_{DRI}) differed according to patient diagnosis (i.e. current level of consciousness) and prognosis (i.e. recovery of full consciousness). In one embodiment, a k-means clustering algorithm (k=2) was then conducted to assess the diagnostic and prognostic accuracy of the ARI. In another embodiment, a logistic regression analysis (e.g., Scikit-learn implementation with L2 penalty) was conducted to assess prognostic accuracy. In this example embodiment, the ARI of 10 patients was studied, with the ARI of patient who recovered consciousness three (3) months after EEG being compared to that of patients who did not recover or remained in a chronic disorder of consciousness.

FIG. 3E illustrates four individual examples 320, 322, 324, 326 depicting the alpha network's response (i.e. reconfiguration) to Propofol administration, in accordance with one embodiment. For each case presented, topographic maps of the node degree of alpha EEG networks and matrices of functional connectivity are presented across pre-anesthesia, anesthesia, and post-anesthesia epochs. The standardized values of the hub and dPLI reconfigurations (Hub_{DRI} s and dPLI_{DRI} s, respectively) are depicted in the right column of each case, and the adaptive reconfiguration index (ARI) is indicated in the bottom right corner of each case. Case 3 (labelled 320), who had acute unresponsive wakefulness syndrome (UWS), showed strong reconfiguration of hubs and dPLI (high adaptive reconfiguration index) and recovered full consciousness within 90 days of the study. Case 7 (labelled 322), who had acute unresponsive wakefulness syndrome, showed an absent reconfiguration to propofol anesthesia (low adaptive reconfiguration index) and did not recover consciousness at follow-up. Case 8 (labelled 324), who had chronic unresponsive wakefulness syndrome, showed a minimal response to propofol, with a pathological response in the post-anesthesia recording (low adaptive reconfiguration index). This patient did not recover consciousness at follow-up. Case 4 (labelled 326) was in an acute coma and had life-sustaining treatment withdrawn. Within 48 hours of withdrawal of treatment, the attending physician indicated a suspicion of complete locked-in syndrome (LIS) and potentially preserved awareness. Although the diagnosis of locked-in syndrome was not confirmed, this patient showed a strong reconfiguration to Propofol (high adaptive reconfiguration index), which is consistent with the clinical suspicion of complete locked-in syndrome.

As seen in FIG. 3E, in the three patients who later recovered full consciousness, the network hub topography mirrored that of healthy individuals (anterior during exposure to Propofol and posterior otherwise) (e.g., see plot 320 of FIG. 3E). In the same three patients who later recovered consciousness, the directed functional connectivity patterns also paralleled those of healthy individuals (feedforward-dominant or neutral dPLI during exposure to Propofol and feedback-dominant dPLI otherwise) (e.g., see plot 320 of FIG. 3E). In contrast, patients who did not recover full consciousness within the follow-up period showed minimal hub reconfiguration during propofol exposure (e.g., see plot 322 of FIG. 3E), or random, incoherent shifts in hub structure that did not return to baseline configuration during the post-anesthesia recording (e.g., see plot 324 of FIG. 3E). The same patients who did not recover consciousness also showed little to no reconfiguration in directed functional connectivity in response to propofol or pathological patterns (e.g., see plots 322 and 324 of FIG. 3E).

FIG. 3F shows a plot 330 illustrating the adaptive reconfiguration index value per patient, in accordance with one embodiment. Individual adaptive reconfiguration index values are depicted as diamonds for acute patients and circles for chronic patients. Patients are organized by outcome at 90-day follow-up, indicated at the bottom of the x-axis. Patients who recovered full consciousness had an adaptive reconfiguration index value above 0, whereas patients who did not recover full consciousness had an adaptive reconfiguration index value below 0. Patient 4 had life-sustaining treatment withdrawn, with a suspicion of complete locked-in syndrome prior to treatment withdrawal. Patient 5 had no post-anesthesia recording and could not be included in the adaptive reconfiguration index calculation.

FIG. 3G shows three plots 340, 342, 344 which illustrate that, in one embodiment, the adaptive reconfiguration index was significantly higher in patients who later recovered consciousness. Hub reconfiguration (labelled Hub_{DRI} on plot 340), directed phase lag index reconfiguration (labelled dPLI_{DRI} on plot 342), and adaptive reconfiguration index (see plot 344) values are depicted per group. Patients who recovered full consciousness within 90 days of the study constitute the “Recovered” group, whereas those who did not recover full consciousness within 90 days constitute the “Did not recover” group. One-tailed Mann-Whitney U test results showed higher Hub_{DRI}, dPLI_{DRI}, and adaptive reconfiguration index in the recovered group, indicating that patients in the recovered group had higher Hub_{DRI} and dPLI_{DRI} values when these indices were taken separately and higher adaptive reconfiguration index values, indicating stronger reconfiguration to Propofol perturbation. Results were statistically significant at P<0.025 for Hub_{DRI} (one-tailed P=0.008) and the adaptive reconfiguration index (one-tailed P=0.008), and showed a trend toward significance for dPLI_{DRI} (one-tailed P=0.033). On plot 342, the single asterisk (*) represents P<0.05 and on plots 330 and 334, the double asterisk (**) represents P<0.025.

FIG. 3H further shows a plot 350 of the standardized reconfiguration of hubs (y-axis) and the standardized reconfiguration of the directed phase lag index (x-axis) per participant in a two-dimensional feature space, yielding the adaptive reconfiguration index, in accordance with one embodiment. Adaptive reconfiguration index value per participant is depicted with circles (“Did not recover full consciousness”) and crosses (“Recovered full consciousness”) according to recovery status at 90-day follow-up. The logistic regression decision boundary (dashed line) separates both groups according to their 90-day outcome. In FIG. 3H, NPV refers to negative predictive value, PPV refers to positive predictive value, and ROC AUC refers to the area under the receiver operating characteristic curve.

From FIGS. 3E to 3H, it can be seen that, on an individual level, a high adaptive reconfiguration index was indicative of favorable prognosis (see FIGS. 3E and 3F). When taken separately, the Hub_{DRI} and dPLI_{DRI} were higher in patients who later recovered full consciousness than in those who did not, reaching statistical significance for Hub_{DRI} (Hub_{DRI} U value=21, one-tailed P=0.008; dPLI_{DRI} U value=19, one-tailed P=0.033) (see plots 340 and 342 of FIG. 3G). This indicated greater reconfiguration in response to Propofol in patients with the capacity to recover. Patients who recovered full consciousness could be separated on an individual subject level from those who did not recover. The minimum Hub_{DRI} and dPLI_{DRI} values in recovered patients were above the maximum values of those who did not recover (see plots 340 and 342 of FIG. 3G). The adaptive reconfiguration index was significantly higher in patients who later recovered full consciousness (U value=21, one-tailed P=0.008) (see plot 344 of FIG. 3G). The logistic regression was able to linearly separate patients according to whether they would recover full consciousness with a sensitivity, specificity, positive predictive value, negative predictive value, and accuracy of 100%, and a ROC AUC of 1 (see FIG. 3H). The adaptive reconfiguration index for all chronic patients was low, as expected, reflecting their low likelihood of recovery.

To assess the translational potential of the ARI to a clinical EEG system, the ARI was recalculated with a selection of eighteen (18) EEG electrodes (10-20 placement) across the patients' healthiest hemisphere, and statistical analyses were re-ran. For focal injuries, the healthiest hemisphere may be defined as the least-injured according to the computed tomography (or CT) scan. For diffuse injuries, the healthiest hemisphere may be selected based on the scalp's condition (i.e. side with absence of wounds, patches, drains, etc.) and the left hemisphere may be selected by default if the scalp is intact. Results showed, in one embodiment, that the ARI was higher in patients who later recovered full consciousness and that the ARI may be able to predict 90-day recovery of consciousness with an accuracy of 100%, even with only eighteen (18) electrode channels placed on a single hemisphere.

FIG. 3I shows plots 360, 362, 364, and 366 that illustrate the adaptive reconfiguration index calculated using 18-channel EEG predicts recovery of consciousness within 90 days. Hub reconfiguration (Hub_{DRI}) (see plot 360), directed phase lag index (dPLI) reconfiguration (dPLI_{DRI}) (see plot 362), and adaptive reconfiguration index values (see plot 364) calculated using 18-channel EEG are depicted for patients who recovered full consciousness within 90 days of the study (i.e., “Recovered”) and those who did not recover full consciousness within 90 days (i.e., “Did not recover”). Hub_{DRI} (see plot 360), dPLI_{DRI} (see plot 362), and the adaptive reconfiguration index (see plot 364) were higher in the recovered group. Results were statistically significant at P<0.025 for Hub_{DRI} (one-tailed P=0.008) and the adaptive reconfiguration index (one-tailed P=0.008) and showed a trend toward significance for dPLI_{DRI} (one-tailed P=0.033). As shown in plot 366, the standardized reconfiguration of hubs (y-axis) and the standardized reconfiguration of the dPLI (x-axis) are plotted per participant in a two-dimensional feature space, yielding the adaptive reconfiguration index. In plot 366, the adaptive reconfiguration index value per participant is depicted by circles (“Did not recover full consciousness”) and crosses (“Recovered full consciousness”) according to recovery status 90 days after the study. The logistic regression decision boundary (dashed line) separates both groups according to their 90-day outcome. On plot 362, the single asterisk (*) represents P<0.05 and on plots 360 and 364, the double asterisk (**) represents P<0.025.

Referring now to FIG. 4A, a method 400 for assessing (or determining) a person's (e.g., a patient's) capacity for consciousness will now be described, in accordance with one embodiment. The method 400 comprises, at step 402, obtaining neuroimaging data acquired by a neuroimaging system before, during, and following exposure of a patient to a neurophysiological perturbation agent having anesthetic properties. In one embodiment, the neuroimaging data corresponds to EEG signal(s) acquired by electrode(s). In one embodiment, the neurophysiological perturbation agent is Propofol and the neuroimaging data is obtained in the manner described herein above with reference to FIG. 1, FIG. 2, and FIG. 3A. The next step 404 comprises determining, from the neuroimaging data, a degree of reconfiguration exhibited by a functional brain network of the patient as a result of exposure to the neurophysiological perturbation agent. Step 406 then comprises computing a measure of the capacity for consciousness of the patient based on the degree of reconfiguration exhibited by the functional brain network (as determined at step 404).

Referring now to FIG. 4B, in one embodiment, step 404 comprises computing a first dynamic reconfiguration index of network hubs of the functional brain network (at step 502) and computing a second reconfiguration index of directed functional connectivity of the functional brain network (at step 504). Steps 502 and 504 may be performed in the manner described herein above with reference to FIG. 1, FIG. 2, and FIG. 3B.

Referring now to FIG. 4C, in one embodiment, step 406 comprises standardizing the first dynamic reconfiguration index and the second dynamic reconfiguration index (at step 602) and combining (i.e. computing a sum of) the first dynamic reconfiguration index and the second dynamic reconfiguration index (at step 604). Steps 602 and 604 may be performed in the manner described herein above with reference to FIG. 1, FIG. 2, FIG. 3C, and FIG. 3D.

In one embodiment, the systems and methods proposed herein demonstrate translational potential for acute clinical settings. Indeed, EEG and Propofol anesthesia can be administered at the bedside, with minimal patient distress or contraindications. EEG is also significantly less expensive than other imaging technologies that may be used for consciousness diagnosis (i.e. current level of consciousness) and prognosis (i.e. recovery of full consciousness). In addition, the proposed systems and methods do not require DOC patients to perform any sensory, motor or cognitive tasks, and are thus independent of the patients' capability for or willingness to react to external stimuli or commands. In addition, the proposed systems and methods do not rely on statistical comparisons between the neurophysiological data of pathologically unresponsive patients and conscious, responsive individuals. Rather, it is proposed herein to employ a within-subject design that is sensitive to the particular neural activity associated with consciousness in each brain-injured individual. In addition, the ARI is calculated from the difference matrices of node degree and directed phase-based functional connectivity across anesthesia-exposure conditions, without any transformations aside from standardization, resulting in simplicity and transparency. Moreover, the ARI may be used to accurately predict recovery of consciousness for patients with various aetiologies of brain injury (traumatic brain injury, anoxic brain injury, stroke), and across diagnoses ranging from coma to minimally conscious state (MCS), thus proving applicable across diverse brain-injured populations.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only. The scope is indicated by the appended claims.

What is claimed is:

1. A method for determining a capacity for consciousness of a person, the method comprising:
obtaining neuroimaging data acquired by a neuroimaging system before, during, and following an exposure of the person to a neurophysiological perturbation agent having anesthetic properties;
determining, from the neuroimaging data, a degree of reconfiguration exhibited by a functional brain network of the person as a result of the exposure to the neurophysiological perturbation agent; and
computing a measure of the capacity for consciousness of the person based on the degree of reconfiguration exhibited by the functional brain network.

2. The method of claim 1, wherein the neuroimaging data comprises one or more electroencephalography (EEG) signals acquired by one or more electrodes, using high-density EEG recording.

3. The method of claim 1, wherein the neuroimaging data is acquired by the neuroimaging system using one of electroencephalography (EEG), functional magnetic resonance imaging (fMRI), and functional near infrared spectroscopy (fNIRS).

4. The method of claim 1, wherein determining the degree of reconfiguration exhibited by the functional brain network of the person comprises:
computing a first dynamic reconfiguration index of hubs of the functional brain network; and
computing a second dynamic reconfiguration index of directed functional connectivity of the functional brain network.

5. The method of claim 4, wherein the measure of the capacity for consciousness of the person is computed by summing the first dynamic reconfiguration index and the second dynamic reconfiguration index.

6. The method of claim 5, further comprising, prior to the summing, standardizing the first dynamic reconfiguration index and the second dynamic reconfiguration index into a common data format.

7. The method of claim 6, wherein standardizing comprises, for each of the first dynamic reconfiguration index and the second dynamic reconfiguration index, removing a mean and scaling to unit variance.

8. The method of claim 4, wherein the first dynamic reconfiguration index is computed as:
Hub_{DRI}=Σi(Aᵢ−Bᵢ)+Σᵢ(Cᵢ−Bᵢ)−Σᵢ(Aᵢ−Cᵢ)
where Hub_{DRI} is the first dynamic reconfiguration index, A is a vector of network degree pre-anesthesia, the vector of network degree indicative of a degree of each node in the functional brain network of the person, B is the vector of network degree during anesthesia, C is the vector of network degree post-anesthesia, and i is an index associated with a given one of one or more electrodes of the neuroimaging system.

9. The method of claim 4, wherein the second dynamic reconfiguration index is computed as:
dPLI_{DRI}=ΣᵢΣⱼ(Aᵢⱼ−Bᵢⱼ)+ΣᵢΣⱼ(Cᵢⱼ−Bᵢⱼ)−ΣᵢΣⱼ(Aᵢⱼ−Cᵢⱼ)
where dPLI_{DRI} is the second dynamic reconfiguration index, A is a directed phase lag index (dPLI) matrix pre-anesthesia, B is the dPLI matrix during anesthesia, C is the dPLI matrix post-anesthesia, and i and j are indices associated with individual ones of one or more electrodes of the neuroimaging system.

10. The method of claim 1, wherein the neuroimaging data is acquired before, during, and following the exposure of the person to the neurophysiological perturbation agent comprising one of Propofol, Sevoflurane, Isoflurane, Dexmedetomidine, and Ketamine.

11. A system for determining a capacity for consciousness of a person, the system comprising:
a processing unit; and
a non-transitory computer-readable memory communicatively coupled to the processing unit and comprising computer-readable program instructions executable by the processing unit for:
obtaining neuroimaging data acquired by a neuroimaging system before, during, and following an exposure of the person to a neurophysiological perturbation agent having anesthetic properties;
determining, from the neuroimaging data, a degree of reconfiguration exhibited by a functional brain network of the person as a result of the exposure to the neurophysiological perturbation agent; and
computing a measure of the capacity for consciousness of the person based on the degree of reconfiguration exhibited by the functional brain network.

12. The system of claim 11, wherein the neuroimaging data comprises one or more electroencephalography (EEG) signals acquired by one or more electrodes, using high-density EEG recording.

13. The system of claim 11, wherein the neuroimaging data is acquired by the neuroimaging system using one of electroencephalography (EEG), functional magnetic resonance imaging (fMRI), and functional near infrared spectroscopy (fNIRS).

14. The system of claim 11, wherein the computer-readable program instructions are executable by the processing unit for determining the degree of reconfiguration exhibited by the functional brain network of the person comprising:
computing a first dynamic reconfiguration index of hubs of the functional brain network; and
computing a second dynamic reconfiguration index of directed functional connectivity of the functional brain network.

15. The system of claim 14, wherein the computer-readable program instructions are executable by the processing unit for computing the measure of the capacity for consciousness of the person by summing the first dynamic reconfiguration index and the second dynamic reconfiguration index.

16. The system of claim 15, further wherein the computer-readable program instructions are executable by the processing unit for, prior to the summing, standardizing the first dynamic reconfiguration index and the second dynamic reconfiguration index into a common data format.

17. The system of claim 14, wherein the computer-readable program instructions are executable by the processing unit for computing the first dynamic reconfiguration index as:
Hub_{DRI}=Σᵢ(Aᵢ−Bᵢ)+Σᵢ(Cᵢ−Bᵢ)−Σᵢ(Aᵢ−Cᵢ)
where Hub_{DRI} is the first dynamic reconfiguration index, A is a vector of network degree pre-anesthesia, the vector of network degree indicative of a degree of each node in the functional brain network of the person, B is the vector of network degree during anesthesia, C is the vector of network degree post-anesthesia, and i is an index associated with a given one of the one or more electrodes.

18. The system of claim 14, wherein the computer-readable program instructions are executable by the processing unit for computing the second dynamic reconfiguration index as:
dPLI_{DRI}=ΣᵢΣⱼ(Aᵢⱼ−Bᵢⱼ)+ΣᵢΣⱼ(Cᵢⱼ−Bᵢⱼ)−ΣᵢΣⱼ(Aᵢⱼ−Cᵢⱼ)
where dPLI_{DRI} is the second dynamic reconfiguration index, A is a directed phase lag index (dPLI) matrix pre-anesthesia, B is the dPLI matrix during anesthesia, C is the dPLI matrix post-anesthesia, and i and j are indices associated with individual ones of the one or more electrodes.

19. The system of claim 11, wherein the neuroimaging data is acquired before, during, and following the exposure of the person to the neurophysiological perturbation agent comprising one of Propofol, Sevoflurane, Isoflurane, Dexmedetomidine, and Ketamine.

20. A non-transitory computer readable medium having stored thereon program code executable by at least one processor for:
obtaining neuroimaging data acquired by a neuroimaging system before, during, and following an exposure of a person to a neurophysiological perturbation agent having anesthetic properties;
determining, from the neuroimaging data, a degree of reconfiguration exhibited by a functional brain network of the person as a result of the exposure to the neurophysiological perturbation agent; and
computing a measure of the capacity for consciousness of the person based on the degree of reconfiguration exhibited by the functional brain network of the person.
